(19) United States
(12) Patent Application Publication
(10) Pub. No.: US 2010/0204104 A1
(43) Pub. Date: Aug. 12, 2010

(54) FUSION PROTEINS CONTAINING TWO TGF-BETA BINDING DOMAINS OF TGF-BETA TYPE II RECEPTOR

(76) Inventors: Huawei Qiu, Westborough, MA (US); Steven R. Ledbetter, Westborough, MA (US); Sirkka Kyostio-Moore, Ashland, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/663,638

(22) PCT Filed: Dec. 24, 2008
(86) PCT No.: PCT/US2008/066941

§ 371 Date: Feb. 19, 2010

Related U.S. Application Data
(60) Provisional application No. 60/944,403, filed on Jun. 15, 2007.

(51) Int. Cl. A61K 38/16; C07K 14/00; C07H 21/04; C12N 15/63; C12N 5/10; C12P 21/06; A61P 13/12; A61K 31/7088; A61P 35/04; A61P 9/10

(52) U.S. Cl. 514/12; 530/350; 536/23.4; 435/320.1; 435/325; 435/358; 435/69.7; 514/44 R

(57) ABSTRACT

The disclosure provides fusion proteins that contain two TGF-β binding domains of TGF-β type II receptor joined to each other by a linker. For example, the C terminus of the first TGF-β binding domain is joined by a short peptide linker (e.g., a 9-glycine linker) to the N terminus of the second TGF-β binding domain. Despite the proximity of the C terminus of the first domain to N terminus of the second domain, such a fusion protein effectively neutralizes TGF-β, in some cases, similarly to anti-TGF-β antibodies.

This application claims priority to U.S. Patent Application No. 60/944,403 filed on Jun. 15, 2007, which is incorporated herein by reference in its entirety.

The present invention relates to TGF-β antagonists and their uses, including, for example, treating diseases.

Transforming growth factor (TGF)-β is involved in many cellular processes including cell growth, cell differentiation, apoptosis, cellular homeostasis and other cellular functions. Overexpression of TGF-β is associated with a number of pathological processes, including, e.g., fibrosis and cancer. The TGF-β family includes three highly conserved isoforms found in mammals (TGF-β1, TGF-β2, and TGF-β3). TGF-β, as well as other members of the TGF-β superfamily, form ˜25 kDa disulfide-linked homodimers. TGF-β binds to a dimeric type II receptor, which is a serine/threonine kinase. The latter then recruits and phosphorylates a type I receptor. The type I receptor phosphorylates a receptor-regulated SMAD which binds to a coSMAD and enters the nucleus to bind transcriptional promoters, thereby regulating expression of multiple genes. The TGF-β type II receptor can bind the ligand independently, but requires the presence of the type I receptor for signaling. The type III TGF-β receptor (also known as betaglycan) functions as a co-receptor to increase ligand binding to TβRII. For a detailed review on the structural and functional aspects of TGF-β and its receptors, see for example, Oppenheim et al. (eds) Cytokine Reference, Academic Press, San Diego, Calif., 2001, e.g., pp. 719-746 and pp. 1871-1888.

Human TGF-β type II receptor (also known as TβRII, AAT3, FAA3, HNPCC6, MFS2, RIIC, and TAAD2) contains 560 amino acids (Lin et al., Cell, 68(4):775-85 (1992); accession No. NP_{—}001020018) and includes a 136-amino acid N-terminal extracellular domain (ECD; represented by SEQ ID NO:31), followed by a single 20-amino acid transmembrane domain, with the remainder being the intracellular (cytoplasmic) domain. The wild-type human TβRII binds TGF-β1 and TGF-β3 with high affinity, while binding TGF-β2 with lower affinity. A sequence alignment for the ECDs of TβRII from several species is depicted in FIG. 1. In addition, an alternatively spliced isoform of TβRII, termed “type II-B” receptor (TβRII-B), has been identified (Suzuki et al., FEBS Lett., 335:19-22 (1994); accession No. GI:456708). This isoform contains an insert of 26 amino acids in place of valine 9 in SEQ ID NO:31. (The ECD of TGFβRIIB represented by SEQ ID NO:32.)

Many TGF-β antagonists have been made that can bind to and inactivate TGF-β. Known TGF-β antagonists include anti-TGF-β antibodies, such as, for example, 1D11, a mouse monoclonal antibody to TGF-β (see U.S. Pat. No. 5,772,998). Other reported TGF-β antagonists include soluble TGF-β receptors and receptor-Fc fusion proteins, for example, as described by Koteliansky (US Pat. App. Pub. No. 2002/004037); Lin et al. (U.S. Pat. No. 6,001,969), Brunkow et al. (U.S. Pat. No. 6,395,511); Reed (U.S. Pat. No. 5,730,976); Gotwals (US Pat. App. Pub. No. 2005/0203022); and Segarini (U.S. Pat. No. 5,693,607).

Given the proposed stoichiometry of TGF-β binding to TβRII (one dimeric ligand to two receptors), it has been suggested that avidity of dimeric antagonists, such as receptor-Fc fusion proteins or coiled-coil dimers of TβRII ECDs, may contribute to the high affinity of the dimeric antagonists, thus providing for more effective neutralization of TGF-β (see, e.g., Crescenzo et al., J. Mol. Biol., 328:1173-1183 (2003)). The predicted structure of the TGF-β-TβRII complex reveals two symmetrically positioned TGF-β-binding sites on the receptor dimer separated by about 65 Å (Hart et al., Nat. Struct. Biol., 9(3):203-8 (2002), see, e.g., FIG. 3 therein). Thus, it is possible that there exist certain structural requirements for dimeric antagonists, such as a sufficient distal separation of the binding sites and the preservation of the symmetrical orientation of the binding sites.

Thus, there continues to be a need to provide soluble TGF-β receptors that have appropriate avidity and affinity, and which are useful as TGF-β antagonists, for example, in order to use them to treat or prevent diseases in which reduction of TGF-β activity is desirable.

Accordingly, one aspect of the invention provides a fusion protein comprising two TGF-β binding domains of TβRII, wherein the binding domains are joined to each other by a linker (e.g., a short peptide linker). In some embodiments, the C terminus of the first TGF-β binding domain is joined by a short peptide linker to the N terminus of the second TGF-β binding domain. Surprisingly, despite the close proximity of the C terminus of the first TGF-β binding domain to the N terminus of the other domain, such a fusion protein does effectively neutralize TGF-β, in some cases, to a similar extent as anti-TGF-β antibodies and TβRII-Fc fusion proteins.

In some embodiments, the linker consists of 50 or fewer amino acids, while each of the TGF-β binding domains comprises a sequence that is at least 60% identical to amino acids 28-129 of SEQ ID NO:31. In some embodiments, one or both of the binding domains comprise SEQ ID NO:8, for example, as set forth by amino acids 28-129 of SEQ ID NO:31. In illustrative embodiments, the linker is a Gly₉ linker and each of the TGF-β binding domains contains amino acids 1-136 of SEQ ID NO:31, for example, as depicted in SEQ ID NO:9. The fusion proteins of the invention may bind to one, two or all of TGF-β1, -β2, and β3. In nonlimiting illustrative embodiments, such as described in the Examples, the fusion protein binds to TGF-β1 and -β3.

The invention further provides nucleic acids encoding the fusion proteins of the invention (e.g., as set forth in SEQ ID NO:10), vectors comprising such nucleic acids, and host cells comprising such nucleic acids.

In another aspect, the invention provides pharmaceutical compositions comprising the fusion proteins of the invention or nucleic acids encoding the fusion proteins.

In yet another aspect, the invention provides methods of producing the fusion proteins, nucleic acids, host cells, and pharmaceutical compositions of the invention.

The invention also provides methods of treating a mammal in need of TGF-β neutralization with a TβRII TGF-β binding domain fusion protein of the invention. The mammal may, for example, have a renal disease. In particular embodiments, the mammal in of TGF-β neutralization may have diabetic nephropathy, radiation nephropathy, obstructive nephropathy, polycystic kidney disease, medullary sponge kidney, horseshoe kidney, nephritis, glomerulonephritis, nephrosclerosis, nephrocalcinosis, Berger's disease (IgA nephropathy), systemic hypertension, glomerular hypertension, tubulointerstitial nephropathy, renal tubular acidosis, renal tuberculosis, or renal infarction.

In another aspect of the methods of the invention, the mammal in need of TGF-β neutralization may, for example, have cancer. In particular embodiments, the cancer to be treated may be stomach cancer, intestinal cancer, skin cancer, breast cancer, or thyroid cancer. In other embodiments, the cancer is melanoma, bone cancer or lung cancer.

In still another aspect of the methods of the invention, the mammal in need of TGF-β neutralization may have a fibrotic or sclerotic disorder or condition. In particular embodiments, the fibrotic or sclerotic disease or disorder to be treated may be scleroderma, atherosclerosis, liver fibrosis, diffuse systemic sclerosis, pulmonary fibrosis, glomerulonephritis, neural scarring, dermal scarring, lung fibrosis, radiation-induced fibrosis, hepatic fibrosis, or myelofibrosis. In a further aspect of the methods of the invention, the mammal in need of TGF-β neutralization may have bronchopulmonary dysplasia (BPD).

### BRIEF DESCRIPTION OF THE DRAWINGS

FIG. 1 shows a sequence alignment for the extracellular domains (ECDs) of TβRII from several species (human (Hum): SEQ ID NO:31; chimpanzee (Ohm): SEQ ID NO:2; rat: SEQ ID NO:3; dog: SEQ ID NO:4; pig: SEQ ID NO:5; mouse (Mus): SEQ ID NO:6; mink (Mnk): SEQ ID NO:7; and a generic sequence (Gen): SEQ ID NO:8). Asterisks denote conserved amino acids. Bold underlined amino acids exemplify amino acids reported to be not essential for TGF-β binding (e.g., as demonstrated by a deletion analysis see, e.g., Guimond et al., FEBS Letters, 84(456):79-84 (1999)). The generic sequence contains conserved amino acids of a TGF-β binding domain, with the exception of amino acids that have been previously shown to be not essential for TGF-β binding.

FIG. 2 shows the amino acid (SEQ ID NO:9) and nucleotide sequences (SEQ ID NO:10) of the sRII-9Gly fusion protein used in the Examples.

FIG. 3 is a schematic illustrating the construction of the sRII-9Gly fusion protein used in the Examples.

FIG. 4 is a graph showing the levels of circulating TGF-β antagonists (sRII-9Gly and 1D11) in a unilateral ureteral obstruction (UUO) rat model during a two-week administration course as indicated.

FIG. 5 is a scatter plot of the results of a histological evaluation of animals treated with particular TGF-β antagonists (sRII-9Gly or 1D11), PBS or a control antibody (1304) in a UUO rat model following a two-week administration course.

FIGS. 6A-6C are graphs showing the levels of collagen content (FIG. 6A), fibrosis by PicroSirius Red staining (FIG. 6B), and collagen III by DAB staining (FIG. 6C) in UUO rat kidney samples following a two-week administration of particular TGF-β antagonists (1D11 and sRII-9Gly) and in control animals (sham, PBS, and 13C4).

FIGS. 7A and 7B are graphs showing results of transcript analysis in UUO rat kidney samples following a two-week administration of particular TGF-β antagonists (1D11 or sRII-9Gly) and in control animals (sham, PBS, and 13C4), for the following genes: collagen III (FIG. 7A); TGF-β1, -β2, and -β3 (FIG. 7B).

FIG. 8A is a graph of the results of a histological evaluation of UUO rats treated with multiple daily dosing of the TGF-β antagonist sRII-9Gly, or with 3× weekly dosing of 1D11, PBS or a control antibody (13C4), following a two-week administration course.

FIG. 8B is a graph showing the levels of collagen detected by hydroxyproline assay in UUO rat kidney samples following multiple daily dosing of the TGF-β antagonist sRII-9Gly, or with 3× weekly dosing of 1D11, PBS or a control antibody (13C4), in a two-week administration course.

FIG. 9A is a graph of the results of a histological evaluation of UUO mice treated with daily dosing of the TGF-β antagonist sRII-9Gly at 7.5 mg/kg, or with 3× weekly dosing of 1D11 at 5 or 10 mg/kg, PBS or 3× weekly dosing of a control antibody (13C4) as indicated, following a two-week administration course.

FIG. 9B is a graph showing the levels of collagen detected by hydroxyproline (Hyp) assay in UUO mice treated with daily dosing of the TGF-β antagonist sRII-9Gly at 7.5 mg/kg, or with 3× weekly dosing of 1D11 at 5 or 10 mg/kg, PBS or 3× weekly dosing of a control antibody (13C4) as indicated, following a two-week administration course.

FIG. 9C is a graph showing the levels of collagen III mRNA detected by RT-PCR in UUO mice treated with daily dosing of the TGF-β antagonist sRII-9Gly at 7.5 mg/kg, or with 3× weekly dosing of 1D11 at 5 or 10 mg/kg, PBS or 3× weekly dosing of a control antibody (13C4) as indicated, following a two-week administration course.

FIG. 9D is a graph showing the levels of PAI-1 mRNA detected by RT-PCR in UUO mice treated with daily dosing of the TGF-β antagonist sRII-9Gly at 7.5 mg/kg, or with 3× weekly dosing of 1D11 at 5 or 10 mg/kg, PBS or 3× weekly dosing of a control antibody (1304) as indicated, following a two-week administration course.

FIG. 10 is a series of radiographic SPECT/CT images of a mouse injected with 125I-labeled human sRII-Gly—the top panels show standard sensitivity, while the bottom panels show increased sensitivity.

FIGS. 11A and 11B are graphs showing the radiological quantitation of 125I-labeled human sRII-Gly in various tissues, expressed as percent of injected dose or normalized to grams of tissue weight respectively, in liver, heart, kidneys, lungs, spleen, tongue, stomach, small intestine, large intestine, rectum, brain, skin (ears), muscle (leg), blood, and scrotum. Each bar represents an individual animal (n=6).

### BRIEF DESCRIPTION OF THE SEQUENCES

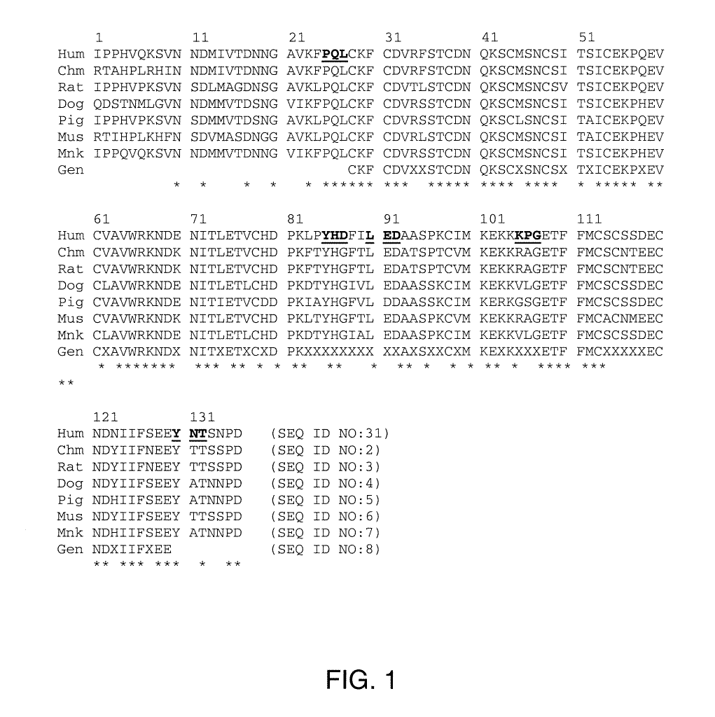

SEQ ID NOs:1-7 provide an amino acid sequence of the ECD of TβRII from the following species: human, chimpanzee, rat, dog, pig, mouse, and mink respectively (FIG. 1). The human sequence (SEQ ID NO:1) encompasses both isoforms, TβRII and TβRIIB, as reflected by variance at valine 9.

SEQ ID NO:8 provides an exemplary genericized amino acid sequence of a minimal TGF-β binding domain of TβRII (FIG. 1).

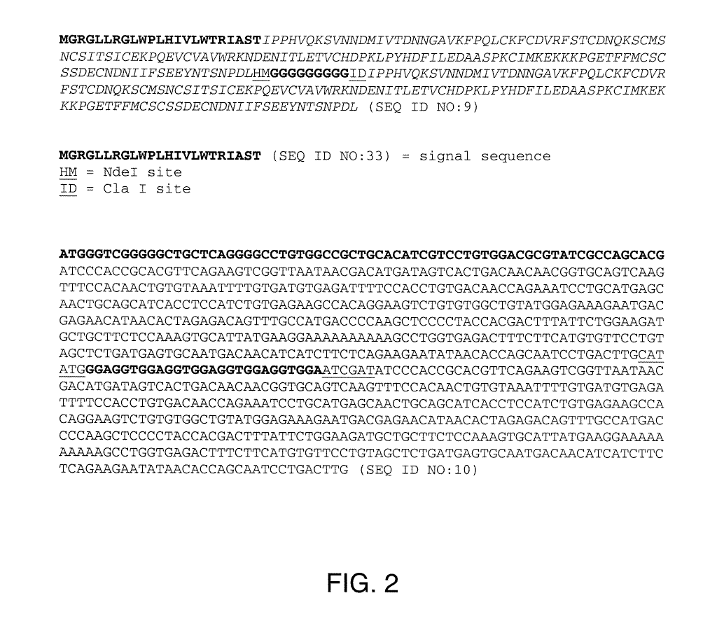

SEQ ID NOs:9-10 provide an amino acid and nucleotide sequences of the sRII-9Gly fusion protein used in the Examples (FIG. 2).

SEQ ID NO:11 provides an amino acid sequence of a glycine-serine linker.

SEQ ID NOs:12-15 provide primer sequences used for making SRII-9Gly as per the Examples.

SEQ ID NOs:16-30 provide primer and probe sequences used for the transcript analysis described in the Examples.

SEQ ID NO:31 provides an amino acid sequence of the ECD of human TβRII, while SEQ ID NO:32 provides an amino acid sequence of the ECD of its TβRIIB isoform.

SEQ ID NO:33 provides an amino acid sequence of the signal sequence depicted in FIG. 2.

### Fusion Proteins

This invention provides fusion proteins that comprise two TGF-β binding domains of TβRII that are joined to each other by a linker, such as, e.g., a peptide linker. The invention is based, in part, on the finding that sRII-9Gly, a soluble form of TβRII constructed by joining two ECDs together with a short peptide linker (Gly9), effectively neutralized TGF-β in cell-based assays. In further in vivo studies in a rat UUO model of kidney disease, treatment with sRII-9Gly resulted in reduced kidney fibrosis and a better preserved kidney morphology. The data indicate that a TGF-β antagonist, such as sRII-9Gly, may effectively neutralize TGF-β. In some embodiments, due to a relatively small size as compared to antibodies and Fc fusion proteins, the fusion protein of the invention may be able to access areas of the body that are not easily accessible by larger molecules. For example, they may pass from the lumen of the glomerular capillary to the urinary space, and subsequently to the tubular epithelial cells, thus achieving a more efficient in situ neutralization of TGF-β than a larger molecule. Thus, in some embodiments, the calculated molecular weight of the fusion protein of the invention is below 100 kDa, 80 kDa, 50 kDa, 40 kDa, or 35 kDa or less, not including post-translational modifications. In the illustrative embodiment, the calculated molecular weight of sRII-9Gly is 32 kDa.

The fusion protein of the invention may bind at least one, two or all of TGF-β1, -β2, and -β3. For example, in some embodiments, the fusion protein binds to TGF-β1 and -β3. Unless stated otherwise, as used herein, the term “TGF-β” refers to at least one TGF-β3 isoform from at least one species but not necessarily all TGF-β isoforms. TGF-β is highly conserved among species. For example, porcine, simian, and human mature TGF-β1 proteins (112 amino acids) are identical, and mouse and rat TGF-β1 proteins differ from human by only one amino acid. Thus, a fusion protein binding to TGF-β from one species is expected to bind to the same isoform from another species. Thus, the fusion protein of the invention may bind to a TGF-β isoform (e.g., TGF-β1 or -β3) with a K_{D} of 10 μM, 1 μM, 500 nM, 100 nM, 10 nM, or lower. In illustrative embodiments, the K_{D} of the fusion proteins of the invention is in the range from 2 to 10 nM. The binding affinity can be measured using, for example, enzyme-linked immunosorbent assays (ELISA) or plasmon resonance (e.g., by Biacore™ as described in the Examples).

The fusion protein of the invention may also inhibit TGF-β binding to a TGF-β receptor thereby neutralizing the biological activity of the TGF-β. In some embodiments, the IC₅₀ of the fusion protein of the invention against one of the TGF-β isoforms (e.g., TGF-β1 or -β3) is 1 μM, 500 nM, 100 nM, 10 nM, 1 nM, 0.1 nM or lower. In illustrative embodiments, the IC50 of the fusion proteins of the invention is in the range from 0.01 to 0.08 nM. Since TGF-β exhibits diverse bioactivities, various assays can be used to detect and quantitate TGF-β neutralizing activity. Examples of some frequently used in vitro bioassays include: (1) induction of colony formation of NRK cells in soft agar in the presence of EGF (Roberts et al., Proc. Natl. Acad. Sci. USA, 78:5339-5343 (1981)); (2) induction of differentiation of primitive mesenchymal cells to express a cartilaginous phenotype (Seyedin et al., Proc. Natl. Acad. Sci. USA, 82:2267-2271 (1985)); (3) inhibition of growth of Mv1Lu mink lung epithelial cells (Danielpour et al., J. Cell. Physiol., 138:79-86 (1989)) or BBC-1 monkey kidney cells (Holley et al., Proc. Natl. Acad. Sci. USA, 77:5989-5992 (1980)); (4) inhibition of mitogenesis of C3H/HeJ mouse thymocytes (Wrann et al., EMBO J., 6:1633-1636 (1987)); (5) inhibition of differentiation of rat L6 myoblast cells (Florini et al., J. Biol. Chem., 261:16509-16513 (1986)); (6) measurement of fibronectin production (Wrana et al., Cell, 71:1003-1014 (1992)); (7) induction of plasminogen activator inhibitor I (PAI-1) promoter fused to a luciferase reporter gene (Abe et al., Anal. Biochem., 216:276-284 (1994)); (8) sandwich ELISA (Danielpour et al., Growth Factors, 2:61-71 (1989)); and (9) A549/IL-11 cell-based assays, as described in the Examples.

### TGF-β Binding Domain

The invention provides fusion proteins containing at least two TGF-β binding domains of TβRII.

TβRIIs from a number of mammalian species have been cloned (e.g., human (Homo sapiens, GI:116242818), chimpanzee (Pan troglydytes, GI:114585822), dog (Canis familiaris, GI:73990406), rat (Rattus norvegicus, GI:207290); pig (Sus scrofa, GI:586087), mouse (Mus muculus, GI:2499656), and mink (Mustela sp., GI:261616)). Unless stated otherwise, as used herein the term “TβRII” and its synonyms refer to a TβRII from at least one mammalian species. As shown in FIG. 1, a sequence alignment for the ECDs of TβRII from seven species demonstrates a high degree of homology. The conserved amino acids are denoted in FIG. 1 by asterisks. The minimal TGF-β binding domain is amino acids 28-129 from any one of SEQ ID NOs:1-7 (see Pepin et al., Biochem. Biophys. Res. Commun., 220:289-293 (1996)). In this region, the sequences exhibit about 70% identity. Deletional analysis has further demonstrated that at least some of the conserved and nonconserved amino acids are not essential for TGF-β binding (see, e.g., Guimond et al., FEBS Letters, 84(456):79-84 (1999)); these amino acids are underlined and bolded in FIG. 1). A generic sequence of the TGF-β binding domain is set forth as SEQ ID NO:8 and contains only the conserved amino acids less the known nonessential amino acids. This genericized sequence is at least 64% identical to any of the seven sequences. It will be understood that further modifications can be made to SEQ ID NO:8 in variable as well as nonvariable amino acid positions without loss of TGF-β binding. Such variants of the TGF-β type II receptor are encompassed within the meaning of the term “TGF-β binding domain” for the purposes of this invention. Such variants may be made by altering the amino acid sequences by substitutions, additions, and/or deletions/truncations that result in functionally equivalent molecules. In general, it may be preferable to substitute or delete amino acids that are positioned in the regions of the molecule that are remote from the surface interacting with TGF-β. The following references provide additional guidance for mutating or deleting amino acid residues in the extracellular domain of TβRII: Lu et al., Cancer Res., 56(20): 4595-98 (1996); Ogasa et al., Gene, 181(1-2):185-90 (1996); Hart et al., Nat. Struct. Biol., 9(3):203-8 (2002); Boesen et al., Structure, 10(7):913-19 (2002); Deep et al., Biochemistry, 42(34):10126-39 (2003); Guimond et al., FEBS Letters, 84(456):79-84 (1999); Tanaka et al., Br. J. Cancer, 82(9):1557-60 (2000); Lucke et al., Cancer Res. 61(2):482-5 (2001); Pannu et al., Circulation, 112(4):513-20 (2005); Mizuguchi et al., Nat. Genet., 36(8):855-60 (2004); Pepin et al., Biochem. Biophys. Res. Commun., 220:289-293 (1996); Loeys et al., Nat. Genet., 37(3):275-81 (2005). Typically, amino acids may be substituted with similar amino acids, i.e., conservative substitutions, without significant loss of function. Substitutes for an amino acid may be selected from other members of the class to which the amino acid belongs, for example, as shown in Table 1. The nonpolar amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Furthermore, many native residues may also be substituted with alanine or glycine.

TABLE 1
Exemplary Amino Acid Substitutions

| | Exemplary | Typical |
|---|---|---|
| Original Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, 1,4-Diamino-butyric Acid, Gln, | Arg |
| | Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, Norleucine | Leu |

Accordingly, in some embodiments, each of the TGF-β binding domains comprises a sequence that is at least 60%, 65%, 70%, 75%, 80%, 85%, 90%, 95%, 97%, or more identical to amino acids 28-129 of any one or more of the following sequences: a) SEQ ID NO:1; b) SEQ ID NO:2; c) SEQ ID NO:3; d) SEQ ID NO:4; e) SEQ ID NO:5; f) SEQ ID NO:6; g) SEQ ID NO:7; and h) SEQ ID NO:31, or to amino acids 53-154 SEQ ID NO:32. In some embodiments, each of the TGF-β binding domains comprises a fragment of any one of the following sequences: a) SEQ ID NO:1; b) SEQ ID NO:2; c) SEQ ID NO:3; d) SEQ ID NO:4; e) SEQ ID NO:5; f) SEQ ID NO:6; g) SEQ ID NO:7; h) SEQ ID NO:31; and i) SEQ ID NO:32, provided that the fragment binds to TGF-β. In some embodiments, each of the TGF-β binding domains comprises amino acids 28-129 of any one of the amino acid sequences set forth in: a) SEQ ID NO:1; b) SEQ ID NO:2; c) SEQ ID NO:3; d) SEQ ID NO:4; e) SEQ ID NO:5; f) SEQ ID NO:6; g) SEQ ID NO:7; h) SEQ ID NO:31; or fragments thereof encompassing amino acids 28-129 from any one of those sequences, including the full-length sequences of a) through i). The fragments encompassing amino acids 28-129 include, for example, fragments with an N terminus starting at amino acid residue 1, 2, 3, 4, 5, 6, 7, 8, 9, 10, 15, 20, 25, 27, 28 and the C terminus ending at amino acid residue 129, 130, 131, 132, 133, 134, 135, and 136.

Percent identity between two amino acid sequences may be determined by standard alignment algorithms, such as, for example, Basic Local Alignment Tool (BLAST) described in Altschul et al., J. Mol. Biol., 215:403-410 (1990), the algorithm of Needleman et al., J. Mol. Biol., 48:444-453 (1970), or the algorithm of Meyers et al., Comput. Appl. Biosci., 4:11-17 (1988). Such algorithms are incorporated into the BLASTP, and “BLAST 2 Sequences” programs (see www.ncbi.nlm.nih.gov/BLAST). When utilizing such programs, the default parameters can be used. For amino acid sequences, the following settings can be used for “BLAST 2 Sequences”: program BLASTP, matrix BLOSUM62, open gap and extension gap penalties 11 and 1 respectively, gap x_dropoff 50, expect 10, word size 3, filter ON.

In some embodiments, one or both of the first and the second TGF-β binding domains comprise SEQ ID NO:8. In other embodiments, one or both of the first and the second TGF-β binding domains comprise SEQ ID NO:1, or a fragment thereof. In some embodiments, the amino acids sequences of the TGF-β binding domains are identical, while in other embodiments, the amino acid sequences are different.

In some embodiments, particularly when the fusion protein is administered in a pharmaceutical preparation, soluble forms of the fusion protein are preferred. Such soluble forms are generally characterized by the absence of a) a substantial portion of, or all of, the transmembrane domain and b) all of or a substantial portion of the cytoplasmic domain.

In illustrative embodiments, each of the TGF-β binding domains contains amino acids 1-136 of SEQ ID NO:1, for example, as set forth in amino acids 24-310 of SEQ ID NO:9 (i.e., without the signal peptide as in the final product). In more specific embodiments, the fusion protein has the sequence as provided in SEQ ID NO:9. Any suitable signal sequence, many of which are known in the art, can be used.

### Linkers

In some embodiments, the invention provides a fusion protein comprising two TGF-β binding domains joined to each other by a linker, such as, e.g., a short peptide linker. In some embodiments, the C terminus of the first TGF-β binding domain is joined by a short peptide linker to the N terminus of the second TGF-β binding domain. A linker is considered short if it contains 50 or fewer amino acids. In some embodiments, the linker is such that its calculated maximum length is 65, 60, 50, 40 Å or less. Assuming an unstructured peptide unit length per amino acid is −3.8 Å, three, four, five and six glycine residues correspond to linkers with maximum lengths of 11.4, 15.2, 19.0 and 22.8 Å, respectively. It will be understood by a skilled artisan that due to the presence of the secondary structure, the actual unit length can be much shorter than the maximum calculated length.

Most typically, the linker is a peptide linker that contains 50 or fewer amino acids, e.g., 45, 40, 35, 30, 25, 20, 15, 10, 9, 8, 7, 6, 5, 3, 4, 2, or 1 amino acid(s). In general, the sequence of the peptide linker is not present in the native amino acid sequence of TGF-β type II receptor. In various embodiments, the linker does not contain more than any 20, or any 10, or any 5 contiguous amino acids from the native receptor sequences. Typically, the linker will be flexible and allow the proper folding of the joined domains. Amino acids that do not have bulky side groups and charged groups are generally preferred (e.g., glycine, serine, alanine, and threonine). Optionally, the linker protein may additionally contain one or more adaptor amino acids, such as, for example, those produced as a result of the insertion of restriction sites. Generally, there will be no more than 10, 8, 6, 5, 4 adaptor amino acids in a linker.

In some embodiments, the linker comprises one or more glycines, e.g., 1, 2, 3, 4, 5, 6, 7, 8, 9, 10, 11, 12, 15, 18, 24, 25, 26, 27, 28, 29, 30, 31, 32, 33, 34, 35, or more glycines. For example, the linker may consist of (GGG)ₙ, where n=1, 2, 3, 4, 5, 6, 7, 8, 9, etc. and optional adaptor amino acids. In illustrative embodiments, the linker is a glycine linker which comprises nine glycines and four adaptor amino acids, e.g., as set forth in amino acids 161-173 of SEQ ID NO:9 (indicated with underline and bold in FIG. 2). In other embodiments, the linker is a glycine-serine linker which comprises (GGGS)ₙ, where n=1, 2, 3, 4, 5, etc. (SEQ ID NO:11). In view of the results disclosed herein, the skilled artisan will recognize that any other suitable peptide linker can be used in the fusion proteins of the invention, for example, as described in Alfthan et al., Protein Eng., 8:725-731 (1995); Argos, J. Mol. Biol., 211:943-958 (1990); Crasto et al., Protein Eng., 13:309-312 (2000); and Robinson et al., Proc. Natl. Acad. Sci. USA, 95:5929-5934 (1998).

### Nucleic Acids, Vectors, Host Cells

The invention further provides nucleic acids encoding any of the fusion proteins of the invention, vectors comprising such nucleic acids, and host cells comprising such nucleic acids. For example, in an illustrative embodiment, the nucleic acid of the invention comprises the sequence as set forth in SEQ ID NO:10 from nucleotide 70 to nucleotide 930 (i.e., without the specific signal sequence) or from nucleotide 1 to 930 of SEQ ID NO:10 (i.e., including the specific signal sequence).

Nucleic acids of the invention can be incorporated into a vector, e.g., an expression vector, using standard techniques, e.g., as described in the Examples. The expression vector may then be introduced into host cells using a variety of standard techniques such as liposome-mediated transfection, calcium phosphate precipitation, or electroporation. The host cells according to the present invention can be mammalian cells, for example, Chinese hamster ovary cells, human embryonic kidney cells (e.g., HEK 293), HeLa S3 cells, murine embryonic cells, or NSO cells. However, non-mammalian cells can also be used, including, e.g., bacteria, yeast, insect, and plant cells. Suitable host cells may also reside in vivo or be implanted in vivo, in which case the nucleic acids could be used in the context of in vivo or ex vivo gene therapy.

### Methods of Making

The invention also provides methods of producing a) fusion proteins, b) nucleic acid encoding the same, and c) host cells and pharmaceutical compositions comprising either the fusion proteins or nucleic acids. For example, a method of producing the fusion protein according to the invention comprises culturing a host cell, containing a nucleic acid that encodes the fusion protein of the invention under conditions resulting in the expression of the fusion protein and subsequent recovery of the fusion protein. In the illustrative embodiments, the fusion protein is expressed in CHO or HEK 293 cells and purified from the medium using methods described in the Examples. In some embodiments, the fusion protein is eluted from a column at a neutral pH or above, e.g., pH 7.5 or above, pH 8.0 or above, pH 8.5 or above, or pH 9.0 or above.

The fusion proteins, including variants, as well as nucleic acids encoding the same, can be made using any suitable method, including standard molecular biology techniques and synthetic methods, for example, as described in the following references: Maniatis (1990) Molecular Cloning, A Laboratory Manual, 2nd ed., Cold Spring Harbor Laboratory, Cold Spring Harbor, N.Y., and Bodansky et al. (1995) The Practice of Peptide Synthesis, 2nd ed., Spring Verlag, Berlin, Germany), or as described in the Examples. Pharmaceutical compositions can also be made using any suitable method, including for example, as described in Remington: The Science and Practice of Pharmacy, eds. Gennado et al., 21th ed., Lippincott, Williams & Wilkins, 2005).

### Pharmaceutical Compositions and Methods of Administration

The invention provides pharmaceutical compositions comprising the fusion proteins of the invention or nucleic acids encoding the fusion proteins.

The fusion protein may be delivered to a cell or organism by means of gene therapy, wherein a nucleic acid sequence encoding the fusion protein is inserted into an expression vector which is administered in vivo or to cells ex vivo which are then administered in vivo, and the fusion protein is expressed therefrom. Methods for gene therapy to deliver TGF-β antagonists are known (see, e.g., Fakhrai et al., Proc. Nat. Acad. Sci. USA, 93:2909-2914 (1996) and U.S. Pat. No. 5,824,655).

The fusion protein may be administered to a cell or organism in a pharmaceutical composition that comprises the fusion protein as an active ingredient. Pharmaceutical compositions can be formulated depending upon the treatment being effected and the route of administration. For example, pharmaceutical compositions of the invention can be administered orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes. The pharmaceutical composition will typically comprise biologically inactive components, such as diluents, excipients, salts, buffers, preservants, etc. Standard pharmaceutical formulation techniques and excipients are well known to persons skilled in the art (see, e.g., Physicians' Desk Reference (PDR) 2005, 59th ed., Medical Economics Company, 2004; and Remington: The Science and Practice of Pharmacy, eds. Gennado et al. 21th ed., Lippincott, Williams & Wilkins, 2005).

Generally, the fusion protein of the invention may be administered as a dose of approximately from 1 μg/kg to 25 mg/kg, depending on the severity of the symptoms and the progression of the disease. The appropriate therapeutically effective dose of an antagonist is selected by a treating clinician and would range approximately from 1 μg/kg to 20 mg/kg, from 1 μg/kg to 10 mg/kg, from 1 μg/kg to 1 mg/kg, from 10 μg/kg to 1 mg/kg, from 10 μg/kg to 100 μg/kg, from 100 μg to 1 mg/kg, and from 500 μg/kg to 5 mg/kg. Effective dosages achieved in one animal may be converted for use in another animal, including human, using conversion factors known in the art (see, e.g., Freireich et al., Cancer Chemother. Reports, 50(4):219-244 (1996)).

The fusion proteins of the invention may be co-administered with other therapeutics, e.g., ACE inhibitors as described in Intl Pat. Appl. Pub. No. WO 04/098637.

### Therapeutic and Non-Therapeutic Uses

The fusion proteins of the invention may be used to capture or neutralize TGF-β, thus reducing or preventing TGF-β binding to naturally occurring TGF-β receptors.

The invention includes a method of treating a mammal by administering to the mammal a fusion protein of the invention or a nucleic acid encoding the fusion protein or cells containing a nucleic acid encoding the fusion protein. The mammal can be for example, primate (e.g., human), rodent (e.g., mouse, guinea pig, rat), or others (such as, e.g., dog, pig, rabbit).

The mammal being treated may have or may be at risk for one or more conditions associated with an excess of TGF-β for which a reduction in TGF-β levels may be desirable. Such conditions include, but are not limited to, fibrotic diseases (such as glomerulonephritis, neural scarring, dermal scarring, pulmonary fibrosis (e.g., idiopathic pulmonary fibrosis), lung fibrosis, radiation-induced fibrosis, hepatic fibrosis, myelofibrosis), peritoneal adhesions, hyperproliferative diseases (e.g., cancer), burns, immune-mediated diseases, inflammatory diseases (including rheumatoid arthritis), transplant rejection, Dupuytren's contracture, and gastric ulcers.

In certain embodiments, the fusion proteins, nucleic acids and cells of the invention are used to treat diseases and conditions associated with the deposition of extracellular matrix (ECM). Such diseases and conditions include, but are not limited to, systemic sclerosis, postoperative adhesions, keloid and hypertrophic scarring, proliferative vitreoretinopathy, glaucoma drainage surgery, corneal injury, cataract, Peyronie's disease, adult respiratory distress syndrome, cirrhosis of the liver, post myocardial infarction scarring, restenosis (e.g., post-angioplasty restenosis), scarring after subarachnoid haemorrhage, multiple sclerosis, fibrosis after laminectomy, fibrosis after tendon and other repairs, scarring due to tatoo removal, biliary cirrhosis (including sclerosing cholangitis), pericarditis, pleurisy, tracheostomy, penetrating CNS injury, eosinophilic myalgic syndrome, vascular restenosis, veno-occlusive disease, pancreatitis and psoriatic arthropathy. In particular, the fusion proteins, and related aspects of the invention are particularly useful for the treatment of peritoneal fibrosis/adhesions. In particular, without being bound to any particular theory, animal studies in rodent models have shown poor systemic bioavailability of the fusion protein in the bloodstream following intraperitoneal administration. In contrast, it is well known that antibodies are readily transferred from the peritoneal cavity into circulation. Therefore, intraperitoneal delivery of the fusion protein may provide a highly localized form of treatment for peritoneal disorders like peritoneal fibrosis and adhesions due to the advantageous concentration of the fusion protein within the affected peritoneum as well as the associated advantage of reduced risk of complications associated with systemic delivery.

The fusion proteins, nucleic acids and cells of the invention are also useful to treat conditions where promotion of re-epithelialization is beneficial. Such conditions include, but are not limited to: diseases of the skin, such as venous ulcers, ischaemic ulcers (pressure sores), diabetic ulcers, graft sites, graft donor sites, abrasions and burns; diseases of the bronchial epithelium, such as asthma and ARDS; diseases of the intestinal epithelium, such as mucositis associated with cytotoxic treatment, oesophagual ulcers (reflex disease), stomach ulcers, and small intestinal and large intestinal lesions (inflammatory bowel disease).

Still further uses of the fusion proteins, nucleic acids and cells of the invention are in conditions in which endothelial cell proliferation is desirable, for example, in stabilizing atherosclerotic plaques, promoting healing of vascular anastomoses, or in conditions in which inhibition of smooth muscle cell proliferation is desirable, such as in arterial disease, restenosis and asthma.

The fusion proteins, nucleic acids and cells of the invention are also useful in the treatment of hyperproliferative diseases, such as cancers including, but not limited to, breast, prostate, ovarian, stomach, renal (e.g., renal cell carcinoma), pancreatic, colerectal, skin, lung, thyroid, cervical and bladder cancers, glioma, glioblastoma, mesothelioma, melanoma, as well as various leukemias and sarcomas, such as Kaposi's Sarcoma, and in particular are useful to treat or prevent recurrences or metastases of such tumors. In particular embodiments, the fusion proteins, nucleic acids and cells of the invention are useful in methods of inhibiting cyclosporin-mediated metastases. It will of course be appreciated that in the context of cancer therapy, “treatment” includes any medical intervention resulting in the slowing of tumor growth or reduction in tumor metastases, as well as partial remission of the cancer in order to prolong life expectancy of a patient. In one embodiment, the invention is a method of treating cancer comprising administering a fusion protein, nucleic acid or cells of the invention. In particular embodiments, the condition is renal cancer, prostate cancer or melanoma.

The fusion proteins, nucleic acids and cells of the invention are also useful for treating, preventing and reducing the risk of occurrence of renal insufficiencies including, but not limited to, diabetic (type I and type II) nephropathy, radiational nephropathy, obstructive nephropathy, diffuse systemic sclerosis, pulmonary fibrosis, allograft rejection, hereditary renal disease (e.g., polycystic kidney disease, medullary sponge kidney, horseshoe kidney), nephritis, glomerulonephritis, nephrosclerosis, nephrocalcinosis, systemic lupus erythematosus, Sjogren's syndrome, Berger's disease, systemic or glomerular hypertension, tubulointerstitial nephropathy, renal tubular acidosis, renal tuberculosis, and renal infarction. In particular embodiments, the fusion proteins, nucleic acids and cells of the invention are combined with antagonists of the renin-angiotensin-aldosterone system including, but not limited to, renin inhibitors, angiotensin-converting enzyme (ACE) inhibitors, Ang II receptor antagonists (also known as “Ang II receptor blockers”), and aldosterone antagonists (see, for example, WO 2004/098637).

The fusion proteins, nucleic acids and cells of the invention are also useful to enhance the immune response to macrophage-mediated infections, such as those caused by Leishmania spp., Trypanosorna cruzi, Mycobacterium tuberculosis and Mycobacterium leprae, as well as the protozoan Toxoplasma gondii, the fungi Histoplasma capsulatum, Candida albicans, Candida parapsilosis, and Cryptococcus neoformans, and Rickettsia, for example, R. prowazekii, R. coronii, and R. tsutsugamushi. They are also useful to reduce immunosuppression caused, for example, by tumors, AIDS or granulomatous diseases.

In certain embodiments, the fusion proteins, nucleic acids and cells of the invention are used to treat diseases and conditions in which a TGF-β antagonist that is smaller in size and/or has a shorter half-life, relative to other TGF-β antagonists, is more effective as a therapeutic agent. As described herein, the fusion proteins of the invention are smaller than other TGF-β antagonists (e.g., TGF-β antibodies, TGF-β receptor-Fc fusion proteins) and have a shorter circulatory half-life. Accordingly, such fusion proteins may show increased efficacy in treating diseases or conditions where such characteristics are desirable. For example, without being bound to any particular theory, it is believed that the fusion proteins of the invention, because of their small size relative to other TGF-β antagonists, may exhibit increased targeting to sites of action (e.g., increased penetration of tumors, increased penetration of tissue (e.g., fibrotic tissue)).

In addition, without being bound to any particular theory, it is also believed that the fusion proteins of the invention, because they lack an immunoglobulin domain (unlike TGF-β antibodies and TGF-β receptor-Fc fusion proteins) may not be as susceptible to clearance from sites of action by the immune system (e.g., in conditions or diseases of the lung).

As described herein and is known in the art, TGF-β is involved in many cellular processes including cell growth, cell differentiation, apoptosis, cellular homeostasis and other cellular functions. Given the crucial role that TGF-β has in cellular processes, there may be conditions or diseases in which it is preferable to administer a shorter-acting TGF-β antagonist, which correspondingly would have fewer negative associated effects than a longer-acting TGF-β antagonist (such as a TGF-β antibody or TGF-β receptor-Fc fusion protein). Accordingly, without being bound to any particular theory, it is believed that the fusion proteins of the invention, because of their shorter circulating half-life, may exhibit fewer negative TGF-β antagonist-related effects. For example, bronchopulmonary dysplasia (BPD), which is a lung disease commonly diagnosed in prenatal infants, may be a condition in which it is beneficial to administer a shorter-acting TGF-β antagonist, such as the fusion proteins of the invention, because such shorter-acting TGF-β antagonists should interfere less with the important role that TGF-β has in normal development.

In some illustrative embodiments, the administration of the fusion proteins of the invention may result in reduced kidney fibrosis and/or better preserved kidney morphology.

The following examples are provided for illustrative purposes and are not intended to be limiting.

### EXAMPLES

### Example 1

### Production and Purification of sRII-9Gly

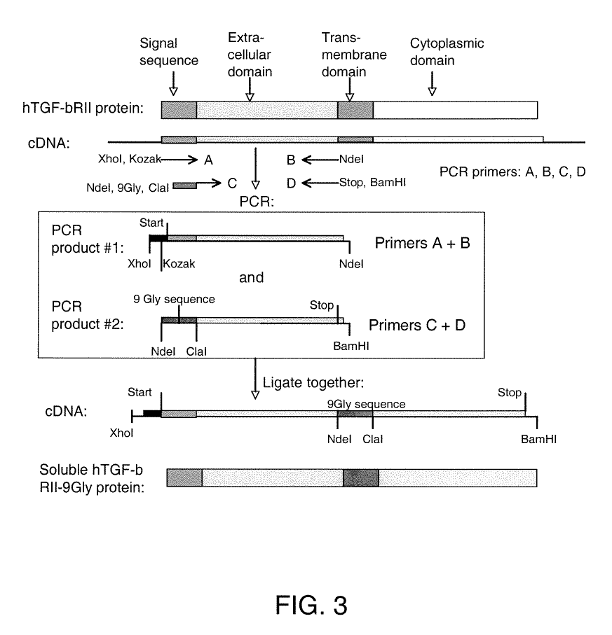

Human TGF-β type II receptor cDNA was used to create a construct for a soluble TGF-β antagonist (sRII-9Gly), containing two ECDs of TβRII joined by a 9Gly linker, as schematically represented in FIG. 3. The primers contained cleavage sites for restriction enzymes and, where necessary, an overlapping sequence for nine glycine residues (Oiu et al., J. Biol. Chem., 273(18):11173-6 (1998)). The first ECD was created using primer A and reverse primer B; the second ECD was created using primer C and reverse primer D as shown in Table 2.

TABLE 2

Primers used for creating the sRII-9Gly construct

| | | |
|---|---|---|
| Primer | Sequences | SEQ ID NO |

| | | |
|---|---|---|
| A | GGCCCTCGAGTCTGCCATGGGTCGGGGGCTG* | SEQ ID NO: 12 |
| B | GGCCCATATGCAAGTCAGGATTGCTGGTGTTATA** | SEQ ID NO: 13 |
| C | GGCCCATATGGGAGGTGGAGGTGGAGGTGGAG | SEQ ID NO: 14 |
| | GTGGAATCGATATCCCACCGCACGTTCAGAAG*** | |
| D | GGCCGGATCCCTACAAGTCAGGATTGCTGGT**** | SEQ ID NO: 15 |

*XhoI site in bold, start codon underlined, Kozak sequence italicized
**NdeI site in bold
***NdeI and ClaI sites in bold, Gly linker underlined
****BamHI in bold; stop codon underlined

The amplified PCR products were digested with respective restriction enzymes and the purified fragments were ligated into the pcDNA3.1 plasmid (Invitrogen, Carlsbad, Calif.), using standard cloning techniques.

The nucleotide sequence of the final sRII-9Gly construct and the corresponding amino acid sequence are set forth in SEQ ID NO:9 and SEQ ID NO:10, respectively. This construct was then subcloned into a CHO expression vector for expression in CHO cells. CHO cell lines expressing the soluble type II receptors were used to produce soluble receptors. CHO production cells lines were thawed and seeded in roller bottles. Production was initiated when cultures were 90% confluent by feeding production medium. Cell medium was collecting at regular intervals with 3-5 harvests from the harvest and supplemented with new medium at the harvest times. In some experiments, sRII-9Gly was produced in HEK 293 cells, using standard protocols. The purification scheme for sRII-9Gly contained three column steps. The first step was primarily a concentration step using anion exchange resin (Pall Q Ceramic HyperD column previously equilibrated with 25 mM HEPES, 0.001% Tween 80, pH 9.0.). The fractions were eluted using 25 mM HEPES, 0.1 M NaCl, 0.001% Tween 80, pH 9.0 and ending with 25 mM HEPES, 0.3 M NaCl, 0.001% Tween 80, pH 9.0. A hydrophobic interaction column followed the primary purification step. The selected fraction(s) from the anion exchange step were titrated to 1.5 M ammonium sulfate, filtered and loaded onto a BioRad MacroPrep Methyl column. Fractions were eluted with 12.5 mM NaPO₄, 0.001% Tween 80, pH 8.0. The selected peak fractions were pooled and then diafiltered using tangential flow filtration into 12.5 mM NaPO₄, 0.001% Tween 80, pH 8.0 to remove any traces of ammonium sulfate that may remain. The last column step was performed with CHT (ceramic hydroxyapatite; BioRad CHT column) and was performed in a flow-through mode to remove impurities. The selected flow-through fractions were formulated to 12 mM NaPO₄, 150 mM NaCl, 0.01% Tween 80, pH 7.2.

### Example 2

### Binding Affinity of sRII-9Gly

The binding affinity of sRII-9Gly produced in CHO and HEK 293 cells was tested using Biacore™. Sensor chips were coated with TGF-β1, -2, or -3. sRII-9Gly was prepared at the concentrations of 1.2, 3.3, 10, 30, and 90 nM in HBS-EP buffer. The protein was injected in duplicates for a 5-minute association followed by a 5-minute dissociation. The resulting binding curves were fitted into a 1:1 binding model. Both CHO- and HEK 293-produced sRII-9Gly bound TGF-β1 and TGF-β3 at a low nM range as shown in Table 3.

TABLE 3
TGF-β binding affinity of CHO-
produced and HEK 293-produced sRII-9Gly

| Production | | Kₒₙ | K_{off} | K_{D} |
|---|---|---|---|---|
| cell line | TGF-β | (×10⁵/ms) | (×10⁻³/s) | (nM) |
| HEK | TGF-β1 | 1.3 | 1.1 | 8.2 |
| 293 | TGF-β2 | no binding | no binding | no binding |
| | TGF-β3 | 1.6 | 0.34 | 2.1 |
| CHO | TGF-β1 | 1.87 | 1.17 | 6.3 |
| (lot 1) | TGF-β3 | 2.64 | 1.31 | 5.0 |
| CHO | TGF-β1 | 1.12 | 1.1 | 9.8 |
| (lot 2) | TGF-β3 | 1.25 | 0.85 | 6.8 |

### Example 3

### Potency Assessment of sRII-9Gly

Potency of HEK 293-produced sRII-9Gly was analyzed using the A549/IL-11 cell bioassay as described in Rapoza et al., J. Immunol. Methods, 316(1-2):18-26 (2006). sRII-9Gly was serially diluted and incubated for 1 hour with a fixed concentration of TGF-β (0.3 ng/ml TGF-β1 or 0.7 ng/ml TGF-β3), after which the samples were transferred onto A549 cells for 18-24 hours. Subsequent hIL-11 levels in the A549 cell supernatants were quantified by ELISA.

The standard curves for TGF-β1 and TGF-β3 generated sigmoid logistic curves with ED₅₀ values of 251 and 281 pg/ml, respectively (typical range of the assay). The IC₅₀ values obtained in this Example are summarized in Tables 4 and 5.

TABLE 4
IC₅₀ values of sRII-9Gly in A549/IL-11 bioassay

| Antagonist vs. TGF-β1 | IC₅₀ (ng/ml) | IC₅₀ (nM) |
|---|---|---|
| sRII-9Gly Lot 1 | 2.55 | 0.075 |
| sRII-9Gly Lot 2 | 2.31 | 0.068 |
| sRII-9Gly Lot 3 | 1.01 | 0.030 |
| sRII-9Gly Lot 4 | 1.37 | 0.040 |

TABLE 5
IC₅₀ values of sRII-9Gly in the A549/IL-11 bioassay

| Antagonist vs. TGF-β3 | IC₅₀ (ng/ml) | IC₅₀ (nM) |
|---|---|---|
| sRII-9Gly Lot 1 | 2.81 | 0.083 |
| sRII-9Gly Lot 2 | 1.54 | 0.045 |
| sRII-9Gly Lot 3 | 0.75 | 0.022 |
| sRII-9Gly Lot 4 | 1.06 | 0.031 |

Among the four lots of sRII-9Gly tested, all exhibited nearly identical potencies against TGF-β1 (within 2-fold) and possessed similar potencies against TGF-β3 (within 4-fold). This data indicates that lots of antagonists generated from the HEK 293 cell line and purified using the same methods generate proteins with similar potency profiles in the A549 bioassay. Some samples of sRII-9Gly eluted above neutral pH (to improve the yield) were tested in the A549 potency assay to see if the pH might have affected their potency. The assay was performed essentially as described above and the results are shown in Table 6.

TABLE 6
IC₅₀ values of sRII-9Gly in the A549/IL-11 cell bioassay

| Antagonist vs. TGF-β1 | IC₅₀ (ng/ml) | IC₅₀ (nM) |
|---|---|---|
| sRII-9Gly Lot 4 | 1.34 | 0.039 |
| sRII-9Gly Lot 5 | 1.80 | 0.053 |
| sRII-9Gly Lot 6 (pH 7.0) | 1.82 | 0.054 |
| sRII-9Gly Lot 7 (pH 8.5) | 2.12 | 0.062 |
| sRII-9Gly Lot 8 (pH 9.0) | 1.59 | 0.047 |

Among the five samples of sRII-9Gly tested, all exhibited nearly identical potencies (in pM range) against TGF-β1, with IC₅₀ values varying by less than 2-fold, (within the variability of the assay). Similar IC₅₀ values were obtained previously for sRII-9Gly lots 1 through 4 as described above. Taken together, these results indicate lots of antagonists generated from the same cell line and purified using similar methods generate proteins with similar potency profiles in the A549 bioassay. Purification of sRII-9Gly within a pH range of 7.0 to 9.0 has no significant effect on its ability to neutralize TGF-β1 in the A549 bioassay.

Potency of CHO-produced sRII-9Gly was analyzed using the A549/IL-11 cell bioassay a similar method. The results are provided in Table 7.

TABLE 7
IC₅₀ (nM) values of CHO-produced sRII-9Gly
in the A549/IL-11 cell bioassay

| Antagonist vs. | | |
|---|---|---|
| TGF-β1 or TGF-β3 | TGF-β1 | TGF-β3 |
| sRII-9Gly Lot 9 | 0.020 | 0.035 |
| sRII-9Gly Lot 10 | 0.018 | 0.290 |
| sRII-9Gly Lot 11 | 0.014 | 0.008 |
| sRII-9Gly Lot 12 | 0.015 | 0.070 |
| sRII-9Gly Lot 13 | 0.016 | 0.018 |

### Example 4

### In Vivo Efficacy of sRII-9Gly

### Two-Week Rat Study

Adult male Sprague-Dawley rats weighing 250 g (7-8 weeks; Taconic Farms, Germantown, N.Y.) were used for the unilateral ureteral obstruction (UUO) model Miyajima et al., Kidney Int., 58(6):2301-13 (2000). All animals were housed in an air-, temperature-, and light-controlled environment. A small ventral midline abdominal incision was made to expose the left kidney and upper ureter, followed by permanent ureteral ligation. Sham operated rats received the same surgical protocol except no ureteral ligation. The rats were dosed as indicated in Table 8. 1D11 is a murine IgG1 monoclonal anti-TGF-β antibody as described in Miyajima et al., supra, or U.S. Pat. Nos. 5,772,998 and 13C4 provides a negative control for 1D11. Blood was collected from retro-orbital sinus into one-tenth volume of 0.105 M sodium citrate on days 3, 7, 15 and 21. Animals were sacrificed two weeks after ligation and the left kidneys were perfused with PBS for 5 minutes and harvested for histological assessment (kidney fibrosis) and transcript analyses.

TABLE 8
Treatment groups (two-week study)

| Grp | Route of | | Animals | |
|---|---|---|---|---|
| # | Delivery | Treatment | per group | Dosing |
| 1 | i.v. | PBS | 8 | N/A 3×/wk |
| 2 | i.v. | sRII-9Gly | 8 | 5 mg/kg daily |
| 3 | i.v. | 1D11 | 8 | 5 mg/kg 3×/wk |
| 4 | i.v. | 13C4 | 8 | 5 mg/kg 3×/wk |
| 5 | i.v. | Sham/PBS | 8 | N/A 3×/wk |

Quantitation of antagonist levels—ELISA assays were developed to quantitate the levels of sRII-(Gly levels in plasma and serum samples. For sRII-9Gly quantitation, the 96 well plates were coated with 100 μl of 1 μg/ml purified TGF-β1 (Genzyme Corp.) in coat buffer (0.1 M NaHCO₃) and incubated overnight at 4° C. The wells were blocked with 150 μl of blocking buffer (0.5% BSA/PBS) for 1 hour. Test samples were added and incubated for 1 hour. Goat anti-human TGF-βRII (Cat. No. AF-241-NA, R&D Systems) was added at 250 ng/ml for 1 hour. Secondary antibody, rabbit anti-goat IgG-HRP conjugate (KPL Cat. No. 14-13-06), was added at 100 ng/ml for 1 hour. OPD substrate (Cat. No. P9187, Sigma, St. Louis, Mo.) was incubated for 15 min in dark followed by addition of 50 μl of 4.5 M H₂SO₄. The color reaction was quantitated by reading the plates at 490 nm with a reference of 650 nm. Purified sRII-9Gly (Genzyme Corp.) was used as a standard. For quantitation of 1D11, the plates were coated with 100 μl of 1 μg/ml purified TGF-β2 (Genzyme Corp., Framingham, Mass.). Blocking was done for 30 minutes at 37° C. Samples were added and incubated for 2 hours at 37° C. followed by an addition of goat anti-mouse IgG-conjugated to HRP (Cat No. P/N A-016, Sigma). Signal was detected using Sigma OPD substrate for 30 min as described above.

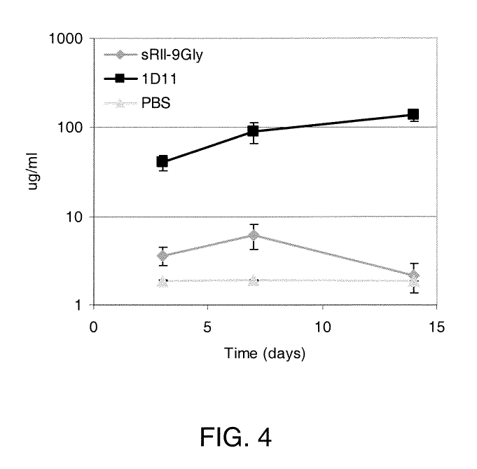

All rats had detectable levels of test compounds by ELISA assay with very little animal-to-animal variation (see FIG. 4). 1D11 levels (half-life of 5-6 days) were on average 100 μg/ml. In contrast, sRII-9Gly was detected below 10 μg/ml despite daily i.v. injections (due to short circulating half-life (18-20 hours)).

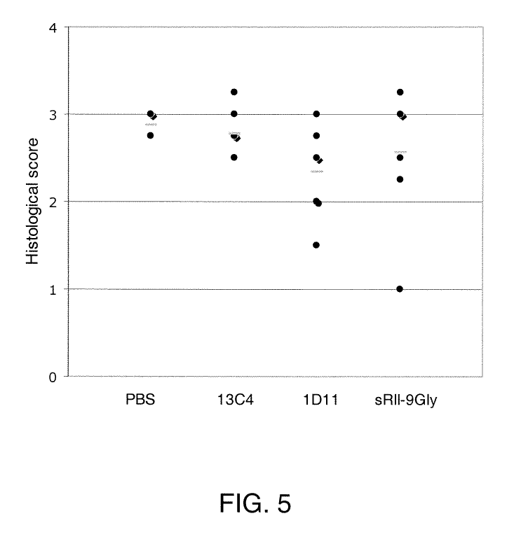

Histopathology analysis—Kidney sections (5-μm) were fixed with 4% paraformaldehyde and embedded in paraffin. The tissue sections were stained with hematoxylin-eosin (H&E) and scored for histopathology in a blinded manner. Histological evaluation performed in a blinded manner showed that 1D11-treated animals had significantly better kidney morphology compared to 13C4/PBS treated animals (p<0.05) as shown in FIG. 5. One animal in the sRII-9Gly group had the best score (1) in the study, but on average, this group was not significantly different compared to the 13C4/PBS-treated animals.

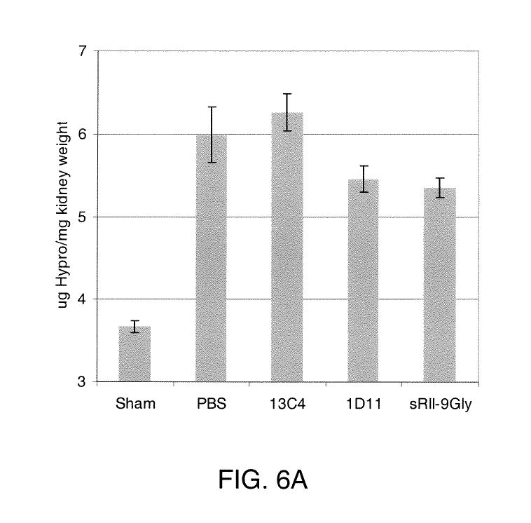

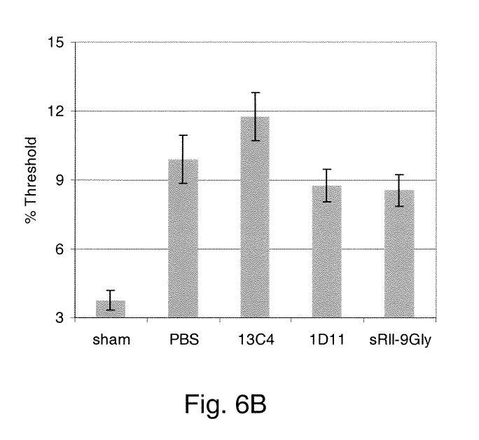

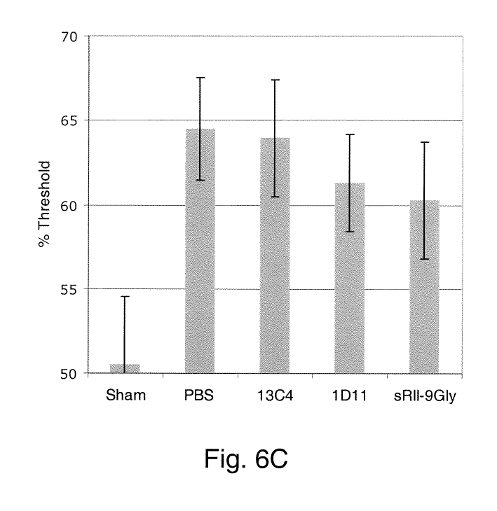

Quantitation of renal fibrosis—Fibrosis was quantitated by hydroxyproline assay and quantitative assessment of Picrosirius red staining and collagen III immunohistochemical staining. Hydroxyproline content was measured as described previously in Kivirikko et al., Physiol. Chem., 348(11):1341-4 (1967) and Reddy et al., Clinical Biochem., 29:225-229 (1996) (modified to a 96-well format). First, kidney tissues were lyophilized in a vacuum lyophilizer and the dried tissue was hydrolyzed in 2 N NaOH (100 μg dry tissue/μl). Ten microliters of sample was added to a 96-well plate and 90 μl of chloramine-T (Sigma) was added to the hydrolyzed samples for 25 min at room temperature. Then 100 μl of Ehrlich's aldehyde (Sigma) was added and samples incubated at 65° C. for 20 min followed by measurement of absorbance at 550 nm. The hydroxyproline content was expressed as μg hydroxyproline per mg of dry tissue. As shown in FIG. 6A, there was a significant reduction in kidney hydroxyproline content in both sRII-9Gly- and 1D11-treated animals (p<0.01 for sRII-9Gly, p<0.05 for 1D11 compared to 13C4).

Kidney sections were stained by Picrosirius red using standard techniques. Images were taken using Chromavision automated software and the staining was quantitated by image analysis using MetaMorph Analysis software (Universal Imaging Corp., Downingtown, Pa.). As shown in FIG. 6B, Picrosirius red-stained collagen was quantified by Metamorph analysis. None of the UUO groups were significantly different compared to PBS-treatment (large variation among animals). However, comparison of sRII-9Gly treatment group to 13C4 was significantly different (p<0.05).

For immunohistochemical detection of collagen III, kidney tissue sections were blocked with 3% peroxide for 10 min, cooked in a pressure cooker with 1% Unmasking solution (Vector Labs) for 7 min followed by a protein block for 30 min. The sections were treated with goat anti-collagen III antibody (1:100)(Southern Biotech) in antibody diluent (Dako) for overnight at 4° C., washed with PBS/0.1% Tween 20 for 5 minutes, followed by treatment with rabbit anti-goat IgG (H+L) conjugated with HRP (1:100; Zymed) in antibody diluent (Dako) for 30 minutes at 37° C. After washing with PBS/0.1% Tween 20 for 5 min the sections were developed in stable diaminobenzine (KPL) for 9 min at room temperature and counterstained with hematoxylin for 30 sec. As shown in FIG. 6C, none of the treatment groups were significantly different compared to the PBS group due to a large variation among the individual animals. However, both sRII-9Gly and 1D11 treatment groups were significantly different from the 13C4-treatment group (p<0.05).

Renal transcript quantitations—Kidney tissue in Trizol (Invitrogen) was homogenized using Zircodia beads (BioSpec) and bead beater, extracted with chloroform and RNA was isolated using RNAeasy kit (Qiagen) according to manufacturer's instructions. The RNA was treated with DNAse I (Promega) and cDNA was prepared using PowerScript plate (BD Biosciences). RT-PCR was set-up using TaqMan™ Universal PCR master mix and gene specific primers (Table 9) and the reactions were run on ABI Prism 7700 Sequence Detector (Perkin Elmer).

TABLE 9
Primers and probes used for renal transcript quantitations

| | | |
|---|---|---|
| TGF-β1 | | |
| Forward | CAAGGTCCTTGCCCTCTACAAC | SEQ ID NO: 16 |
| Reverse | GGCTTGCGACCCACGTAGTA | SEQ ID NO: 17 |
| Probe | CCGCAGGCTTTGGAGCCACTG | SEQ ID NO: 18 |
| TGF-β2 | | |
| Forward | AGGAGTACTACGCCAAGGAGGTT | SEQ ID NO: 19 |
| Reverse | GGACGATTCTGAAGTAGGGTCTGT | SEQ ID NO: 20 |
| Probe | CCCTCCGAAAATGCCATCCCG | SEQ ID NO: 21 |
| TGF-β3 | | |
| Forward | AGCCCCTGACCATCTTGTACTA | SEQ ID NO: 22 |
| Reverse | CGACTTCACCACCATGTTGG | SEQ ID NO: 23 |
| Probe | CAGAACCCCCAAGGTGGAGCAGC | SEQ ID NO: 24 |
| TGF-βRII | | |
| Forward | CTGGACACGCTGGTGGG | SEQ ID NO: 25 |
| Reverse | AAGTGTTCTGCTTCAGCTTGCC | SEQ ID NO: 26 |
| Probe | AAGGGCCGCTTCGCCGAGG | SEQ ID NO: 27 |
| TGF-β RIII | | |
| Forward | TCGATGGAAATGCTACCTTCAA | SEQ ID NO: 28 |
| Reverse | TGTGCCACAGACAAGACCC | SEQ ID NO: 29 |
| Probe | AGACCTCTTTCTGGTGCCCTCCCCA | SEQ ID NO: 30 |
| Collagen III | | |
| | Cat. No. Rn01437683_m1 (Col3a1) | |
| | Gene axpressions assays (Applied | |
| | Biosystems) | |

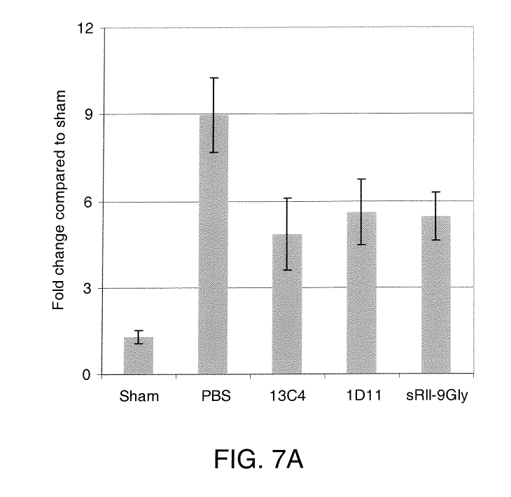

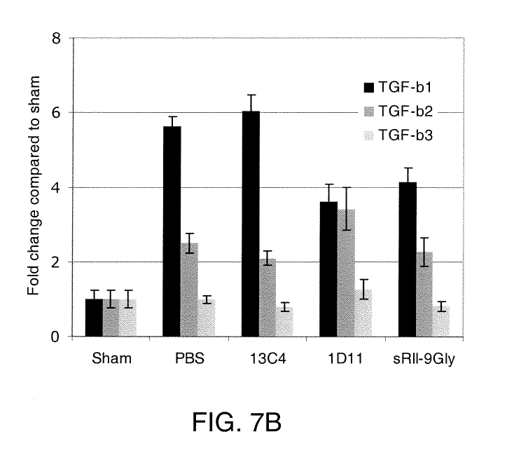

FIG. 7A shows transcript levels for collagen III assessed by RT-PCR. Both 1D11 (p<0.05) and sRII-9Gly (p<0.01) treatments significantly reduced collagen III message levels compared to PBS-treated animals. This data correlated with results on collagen III protein levels by IHC/Metamorph. It is unclear why collagen III mRNA levels were also decreased by 13C4-treatment.

Message levels for each of the TGF-β isoforms were expressed as fold difference in copy numbers (FIG. 7B) compared to sham animals. There were 6- and 2-fold increases in TGF-β1 and 132 mRNA levels, respectively, in the obstructed kidneys compared to sham animals while no changes in TGF-β3 mRNA were observed. As shown in FIG. 7B, among the treatment groups, the TGF-β1 mRNA levels in sRII-9Gly and 1D11-treatment groups were decreased 30 and 40%, respectively, compared to 13C4-treatment (p<0.01). No changes were observed in TGF-β2 and TGF-β3 mRNA levels in any of the antagonist treatment groups. In sham animals TGF-β2 mRNA was significantly higher than TGF-β1 suggesting that TGF-β2 plays a role in normal kidneys (also seen IHC analysis for TGF-β isoforms).

TGF-β neutralizing antibody, 1D11, persisted in the circulation after a 3 time per week dosing schedule. In contrast, sRII-9Gly levels were 10-fold lower due to its short plasma half-life. In this study, both 1D11 and sRII-9Gly reduced kidney fibrosis to a similar extent assessed by various endpoints. Both antagonists slightly improved kidney morphology and reduced the severity of fibrosis as shown by reduced hydroxyproline content, collagen III protein levels and Pircosirius red staining. Furthermore, both sRII-9Gly and 1D11 reduced TGF-β1 (no effect on TGF-β2 or TGF-β3 levels) and collagen III transcript levels. None of the antagonists tested affected endogenous TGF-β receptor (RII, RIII) levels in the obstructed kidneys. Similar efficacy by 1D11 and sRII-9Gly was obtained despite 10-fold lower levels of sRII-9Gly in circulation compared to 1D11. This suggests that sRII-9Gly is more potent than 1D11 or it localizes differentially in the kidneys. The molecular weight of sRII-9Gly is smaller (55-60 kDa with glycosylation) than 1D11 (150 kDa) and this difference could confer better access of sRII-9Gly to the renal urinary space and/or better tissue penetration into the tubulointerstitial space. A study similar to the one described in Example 4 was performed in the same model with a 3-week (vs. 2-week) duration. Poor efficacy of sRII-9Gly was seen in that extended study, most likely due to potential immune response in rats against human soluble receptor protein.

### Example 5

### In Vivo Efficacy of sRII-9Gly in Rat UUO Model

### Multiple Daily Dosing

Adult male Sprague-Dawley rats were used for the unilateral ureteral obstruction (UUO) model in a two-week study similar to that described above in Example 4, except the sRII-9Gly-treatment groups received three doses daily as summarized below.

TABLE 10
Treatment groups (multiple daily dosing rat study)

| Grp | Route of | | Animals | |
|---|---|---|---|---|
| # | Delivery | Treatment | per group | Dosing |
| 1 | | Sham | 6 | |
| 2 | i.v. | PBS | 6 | N/A 3×/wk |
| 3 | i.v. | 13C4 | 6 | 10 mg/kg 3×/wk |
| 4 | i.v. | 1D11 | 6 | 10 mg/kg 3×/wk |
| 5 | i.v. | sRII-9Gly | 6 | 10 mg/kg 3×/day |

The levels of the various forms of TGF-β antagonist in the bloodstreams of the treated rats were assessed as follows. sRII-9Gly levels in blood were assayed 15 min after the last daily dose (post-bleed) and again, after overnight the next day, but before first daily dose (i.e, prebleed) in order to observe peak and trough levels. The levels of sRII-9Gly were comparable to 1D11 right after dosing (150-300 μg/ml); however, the trough levels were lower at 14 to 60 ug/ml.

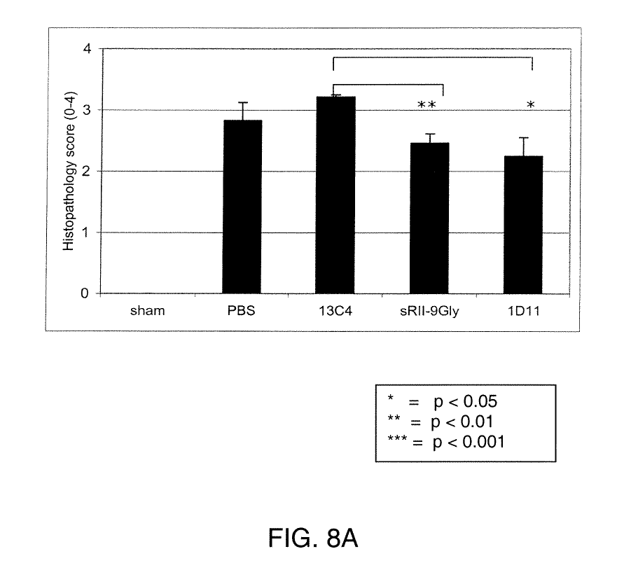

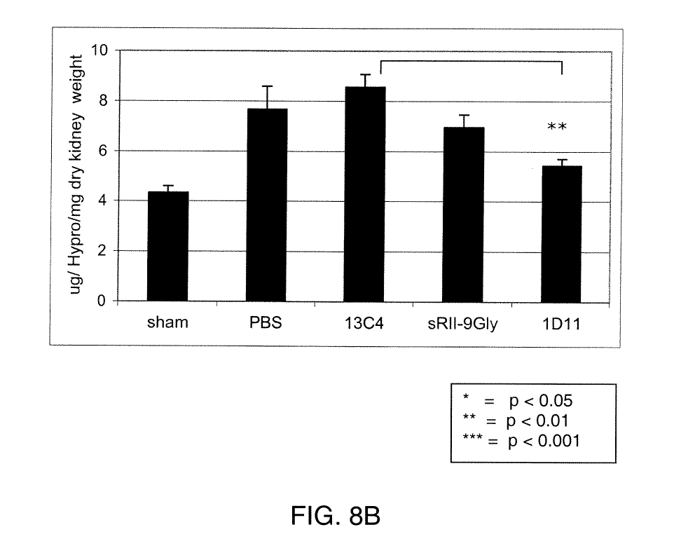

Efficacy endpoints—The efficacy of the antagonists were evaluated by histopathological scoring and quantitation of renal fibrosis similar to analyses in Example 4. The tissue sections were stained with H&E and scored for histopathology in a blinded manner. The H&E-stained kidney sections showed significant preservation of kidney morphology in the sRII-9Gly (p<0.01) and 1D11 (p<0.05) treatment groups compared to the 13C4-treatment group (FIG. 8A). Quantitation of collagen levels by hydroxyproline assay demonstrated significantly lower levels of collagen in 1D11-treated animals (p<001) and a similar trend in the sRII-9Gly-treatment group (FIG. 8B).

In summary, the efficacy of multiple daily dosing of sRII-9Gly observed in the rat UUO model, as assessed by kidney morphology and quantitation of kidney fibrosis, was comparable to that achieved with the 1D11 anti-TGF-β antibody.

### Example 6

### In Vivo Efficacy in Mouse UUO Model

Adult male C57BL/6 mice weighing 25-30 g (10-12 weeks; Charles River Laboratories) were used and surgery was performed similarly to rat model described above. The mice were dosed as indicated in Table 11. The animals were sacrificed two weeks after ureteral ligation and the perfused kidneys were harvested for analysis.

TABLE 11
Treatment groups (daily dosing mouse study)

| Grp | Route of | | Animals | |
|---|---|---|---|---|
| # | Delivery | Treatment | per group | Dosing |
| 1 | i.v. | Sham | 7 | N/A |
| 2 | i.v. | PBS | 9 | 3×/wk |
| 3 | i.v. | 13C4 | 8 | 10 mg/kg 3×/wk |
| 4 | i.v. | 1D11 | 9 | 10 mg/kg 3×/wk |
| 5 | i.v. | 1D11 | 9 | 5 mg/kg daily |
| 6 | i.v. | sRII-9Gly | 9 | 7.5 mg/kg daily |

Levels of detectable sRII-9Gly in the bloodstream were quantitated and found to be 8 and 2 ug/ml on days 7 and 15, respectively. A majority of the sRII-9Gly-treated animals had a drop in detectable blood sRII-9Gly levels despite daily i.v. dosing. This drop may have been due to the generation of neutralizing antibodies against human sRII-9Gly protein, which could have interfered with sRII-9Gly binding to TGF-β1 on the ELISA plate.

Potential antibody generation against human sRII-9Gly was evaluated in day 15 plasma samples. ELISA plates were coated with CHO-produced human sRII-9Gly followed by the addition of day 15 plasma samples. Goat anti-mouse IgG antibody conjugated to HRP was added followed by ODP substrate. The color reaction was quantitated by reading the plates at 490 nm with a reference of 650 nm. The antibody titer was defined as the reciprocal of the dilution giving OD>0.100 (background of the assay was <0.05). The results showed that all nine mice had antibodies against the sRII-9Gly soluble human TGF-β receptor construct, however this assay does not distinguish neutralizing antibodies that interfere with TGF-β binding from non-neutralizing antibodies that do not interfere with TGF-β binding.

The antibody response to the human sRII-9Gly protein is not unexpected in rodents, however the development of antibodies could affect the efficacy of the sRII-9Gly construct, particularly if the antibodies generated compromised the ability of sRII-9Gly to neutralize TGF-β. The low levels of sRII-9Gly detected by ELISA in the circulation of the majority of mice on day 15 compared to day 7 suggest that these antibodies indeed reduced the sRII-9Gly binding to TGF-β, and therefore the UUO animal model may significantly underestimates the efficacy of the human sRII-9Gly as a therapeutic TGF-β antagonist.

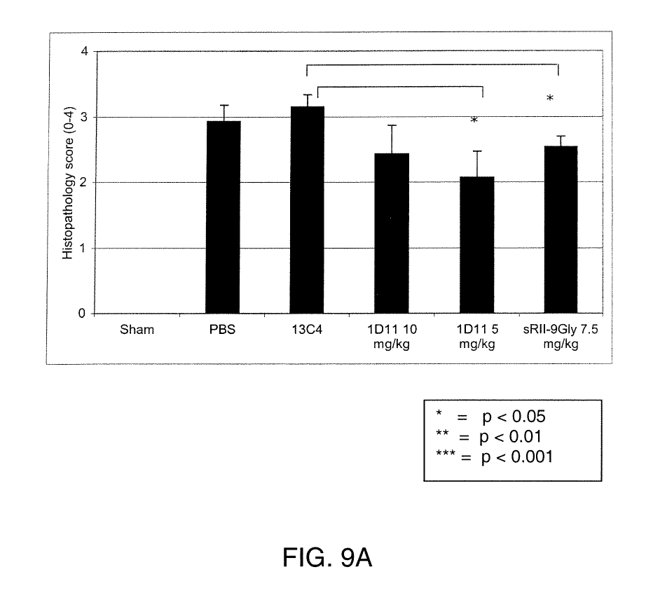

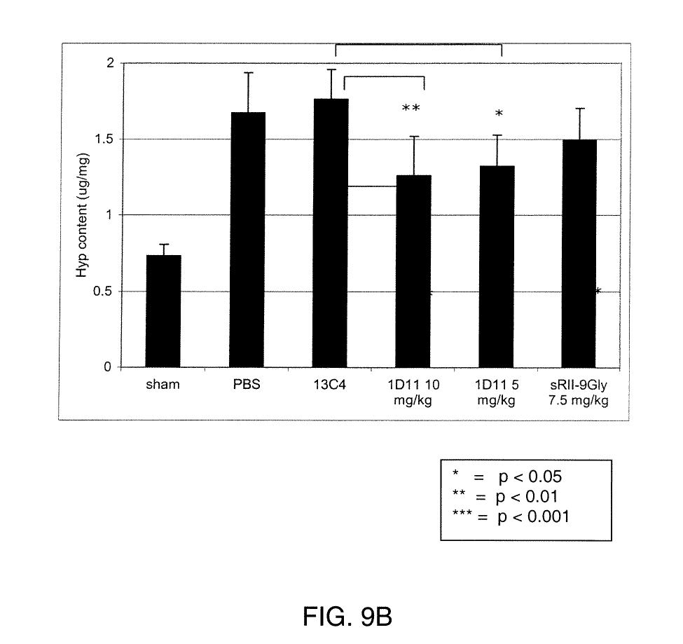

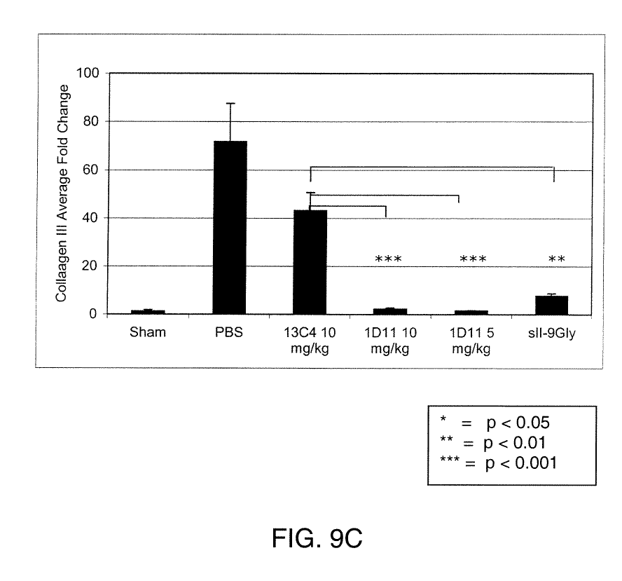

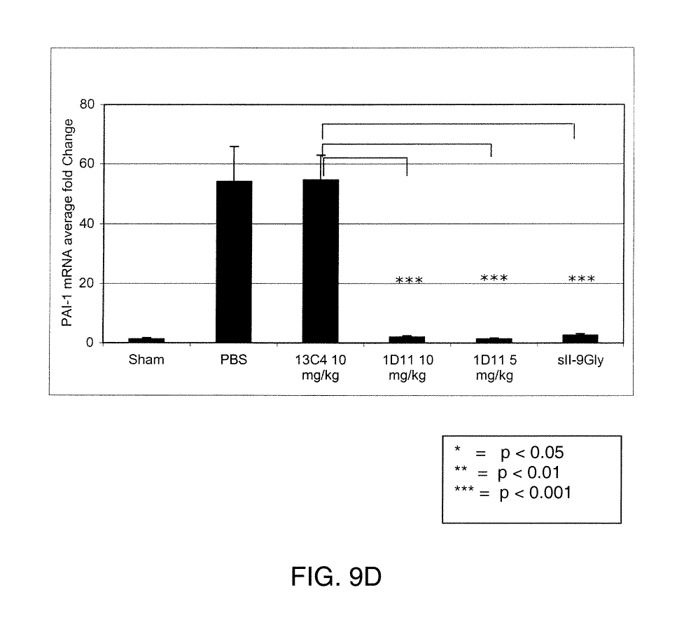

The efficacy of sRII-9Gly in the mouse UUO model was evaluated by histopathological scoring, quantitation of renal fibrosis and transcriptional analysis similar to the analyses performed above in Example 4. H&E-stained kidney sections were scored in a blinded manner and showed significant preservation of kidney morphology in both 1D11 (5 mg/kg) and sRII-9Gly treated animals compared to 13C4 treatment group (p<0.05) (FIG. 9A). Furthermore, quantitation of collagen levels by hydroxyproline assay demonstrated significantly lower levels of collagen in 1D11-treated animals (p<0.01) and similar trend in sRII-9Gly treatment group (FIG. 9B).

Renal transcript levels for collagen III and plasminogen activator inhibitor-1 (PAI-1) were assessed by RT-PCR. Both 1D11 and sRII-9Gly significantly reduced collagen III (p<0.001 for 1D11 and p<0.01 for sRII-9Gly) (FIG. 9C) and PAI-1 mRNA levels (p<0.001 for both 1D11 and sRII-9Gly) (FIG. 9D) compared to PBS- and 13C4-treated animals.

In summary, sRII-9Gly and control antibody 1D11 showed efficacy in the mouse UUO model by preserving kidney morphology and reducing transcription of fibrosis-related genes and subsequent fibrosis.

### Example 7

### Biodistribution of sRII-9Gly in Normal Mice after Single Administration

Human sRII-Gly was labeled with ¹²⁵I using IODO-GEN method (Pierce) and purified using Sephadex G-25 column (Pierce). Six male Balb/c mice were dosed i.v. with 10 mg/kg of sRII-9Gly (200 μg sRII-9Gly including 22 μg of ¹²⁵I-labeled sRII-9Gly with total radioactivity of about 680 μCi per mouse). Selected organs (heart, left and right kidney, lungs, stomach, thyroid, brain, muscle, scrotum and whole body) were scanned at 30 min, 2-3, 8 and 24 hrs post administration using SPECT/CT and region of interest (ROI) counts were calculated. After the last image, mice were sacrificed and selected organs were excised and counted for radioactivity (liver, heart, kidney, lungs, spleen, tongue, stomach, esophagus, small and large intestines, colon, rectum, brain, femur, skin [ear] and muscle). For the excised organs, the counts were normalized with tissue weight.

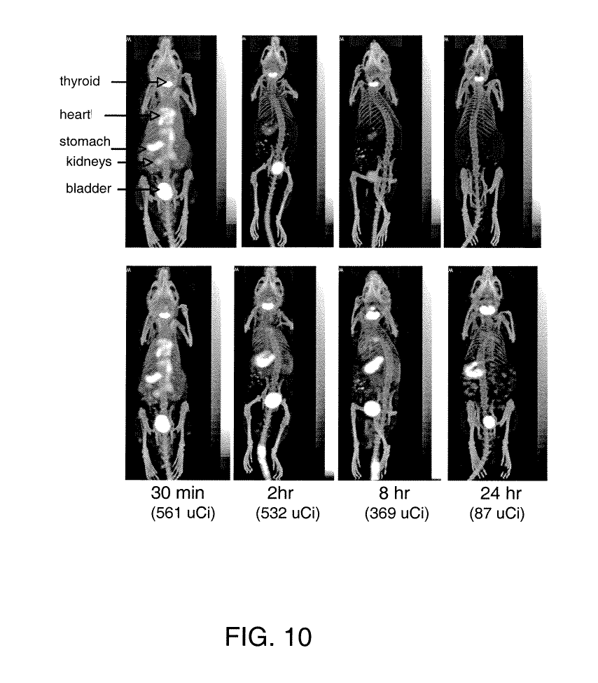

The SPECT/CT images showed sRII-9Gly at 30 min in heart, carotid arteries, both kidneys, stomach and bladder (FIG. 10). There was a decreasing activity with time, indicating a rapid clearance of the sRII-9Gly. A strong sRII-9Gly signal in the bladder, and low levels detected in the liver and spleen, shows that sRII-9Gly clearance occurred primarily via the kidney. At 8 and 24 hrs, 10-47% and 80-85% of the total counts, respectively, had been cleared. Little activity was seen at 24 hr except in the thyroid. Increasing the scale of SPECT/CT images for later timepoints demonstrated sRII-9Gly localization in the same organs throughout the time course (heart, stomach, and bladder).

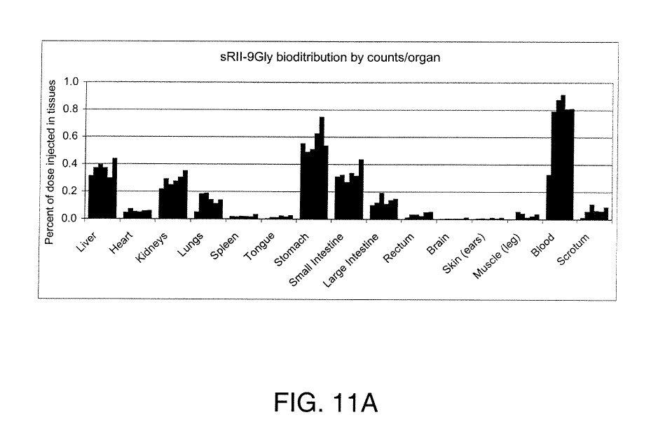

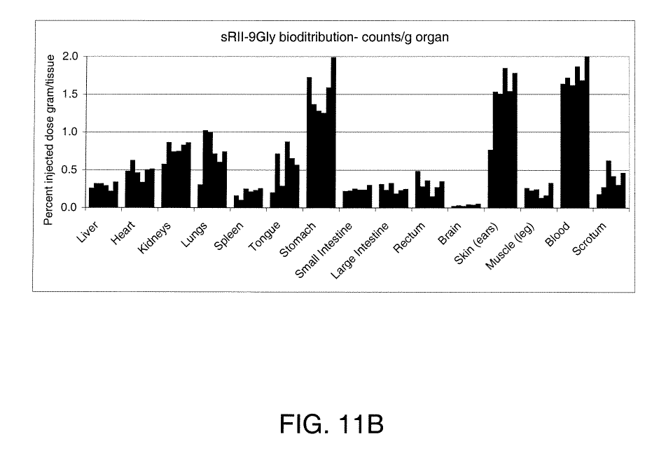

The biodistribution of sRII-9Gly at 24 hrs was also determined by counting radioactivity of the excised organs. This was expressed as 1) percentage of total radioactivity (cpm) in excised tissues compared to activity injected (FIG. 11A) and 2) percentage of the tissue counts normalized by tissue weight (FIG. 11B).

Radioactivity in excised organs was highest in blood, stomach, liver, kidney and small intestine (>0.2%). Normalization by tissue weight indicated the highest amounts of sRII-9Gly in blood, skin and stomach (1.5-1.8%), followed by kidney and lungs (0.7-0.8%), and heart and tongue (0.5%). The remainder of organs analyzed contained 0.2-0.3%, except brain which had only 0.04%.

In summary, the biodistribution of ¹²⁵I-labeled sRII-9Gly was assessed in normal mice by SPECT/CT scan. Both SPECT/CT imaging and quantitation of radioactivity of excised organs were comparable and similar in all animals. As expected, the majority of the test article was cleared within 24 hrs by the kidney, with the remainder of material mostly detectable in blood, skin and stomach. These results suggest sRII-9Gly can be efficiently retained in skin and stomach, as well as the kidney and lungs. Accordingly, the sRII-9Gly may be particularly useful in the treatment of diseases and disorders affecting these tissues and organs where sRII-9Gly is efficiently retained, such as sclerotic and fibrotic diseases and disorders of the skin (such as scleroderma), skin cancers (such as melanoma), renal diseases and disorders (such as diabetic nephropathy), and lung diseases and disorders (such as lung cancer and bronchopulmonary dysplasia).

The specification is most thoroughly understood in light of the teachings of the references cited within the specification which are hereby incorporated by reference. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. The citation of any references herein is as not an admission that such references are prior art to the present invention. The representations of molecular mechanisms and pathways are provided for ease of the understanding of the invention only and should not be considered binding.

1. A fusion protein comprising two TGF-β binding domains of TGF-β receptor II, said binding domains joined to each other by a peptide linker.

2. The fusion protein of claim 1, wherein the C terminus of the first TGF-β binding domain is joined by the linker to the N terminus of the second TGF-β binding domain.

3. The fusion protein of claim 1, wherein the two domains are identical.

4. The fusion protein of claim 1, wherein the linker consists of 50 or fewer amino acids.

5. The fusion protein of claim 4, where the maximum calculated length of the linker is 65 Å.

6. The fusion protein of claim 5, wherein the linker comprises one or more glycines.

7. The fusion protein of claim 5, wherein the linker comprises nine glycine residues.

8. The fusion protein of claim 5, wherein one or both of the TGF-β binding domains comprises a sequence that is at least 60% identical to amino acids 28-136 of SEQ ID NO:31.

9. The fusion protein of claim 5, wherein one or both of the TGF-β binding domains comprises a sequence as set forth in SEQ ID NO:8.

10. The fusion protein of claim 9, wherein one or both of the TGF-β binding domains comprises amino acids 28-129 of SEQ ID NO:31.

11. The fusion protein of claim 1, having a molecular weight less than 100 kDa.

12. The fusion protein of claim 1, wherein the fusion protein is soluble.

13. The fusion protein of claim 1, comprising i) two TGF-β binding domains each having a sequence as set forth in SEQ ID NO:31, ii) a 9Gly linker joining the two domains, and optionally, iii) one or more adaptor amino acids.

14. The fusion protein of claim 1, comprising an amino acid sequence as set forth from amino acid 24 to amino acid 310 of SEQ ID NO:9.

15. The fusion protein of claim 1, comprising an amino acid sequence as set forth in SEQ ID NO:9.

16. A pharmaceutical composition comprising the fusion protein of claim 1.

17. A nucleic acid encoding the fusion protein of claim 1.

18. The nucleic acid of claim 17, comprising the sequence as set forth in SEQ ID NO:10.

19. A vector comprising the nucleic acid of claim 17.

20. A host cell comprising the nucleic acid of claim 17.

21. The host cell of claim 20, wherein the cell is a mammalian cell.

22. The host cell of claim 21, wherein the cell is a CHO or a HEK 293 cell.

23. A method of producing a fusion protein, comprising culturing the host cell of claim 20, and recovering the fusion protein from the cell culture.

24. A method of treating a disease or condition associated with TGF-β expression in a mammal, comprising administering to the mammal a fusion protein of claim 1 or a nucleic acid of claim 17.

25. The method of claim 24, wherein the disease or condition is a renal disease or disorder.

26. The method of claim 25, wherein the renal disease or disorder is selected from the group consisting of diabetic nephropathy, radiation nephropathy, obstructive nephropathy, polycystic kidney disease, medullary sponge kidney, horseshoe kidney, nephritis, glomerulonephritis, nephrosclerosis, nephrocalcinosis, Berger's disease (IgA nephropathy), systemic hypertension, glomerular hypertension, tubulointerstitial nephropathy, renal tubular acidosis, renal tuberculosis, and renal infarction.

27. The method of claim 24, wherein the disease or condition is a cancer.

28. The method of claim 27, wherein the cancer is selected from the group consisting of stomach cancer, intestinal cancer, skin cancer, breast cancer, and thyroid cancer.

29. The method of claim 27, wherein the cancer is melanoma.

30. The method of claim 27, wherein the cancer is bone cancer.

31. The method of claim 27, wherein the cancer is lung cancer.

32. The method of claim 24, wherein the disease or condition is a fibrotic or sclerotic disease or disorder.

33. The method of claim 32, wherein the fibrotic or sclerotic disease or disorder is selected from the group consisting of scleroderma, atherosclerosis, liver fibrosis, diffuse systemic sclerosis, pulmonary fibrosis, glomerulonephritis, neural scarring, dermal scarring, lung fibrosis, radiation-induced fibrosis, hepatic fibrosis, and myelofibrosis.

34. The method of claim 24, wherein the disease or disorder is bronchopulmonary dysplasia.
